(19) United States
(12) Patent Application Publication
(10) Pub. No.: US 2022/0305278 A1
(43) Pub. Date: Sep. 29, 2022

(54) Topical Therapeutics

(71) Applicant: Bruce H. Levin, Oceanside, NY (US)

(72) Inventor: Bruce H. Levin, Oceanside, NY (US)

(21) Appl. No.: 17/841,580

(22) Filed: Jun. 15, 2022

Related U.S. Application Data
(62) Division of application No. 16/551,657, filed on Aug. 26, 2019.
Application No. 16/551,657 is a continuation-in-part of application No. 15/998,669, filed on Aug. 15, 2018.
(60) Provisional application No. 62/722,778, filed on Aug. 24, 2018.

(51) Int. Cl. A61N 2/00; A61N 2/02; A61F 7/03; A61N 7/00; A41D 13/12; A61M 31/00; A61K 9/00; A61K 9/70; A61N 2/06

(52) U.S. Cl. CPC A61N 2/002; A61N 2/02; A61F 7/03; A61N 7/00; A41D 13/1254; A61M 31/00; A61K 9/0014; A61K 9/7007; A61K 9/703; A61K 9/0009; A61N 2/06; A61F 5/01

(57) ABSTRACT

The present invention classifies disorders, including Local Anesthetic Responsive Headaches as a general class. The present invention also uses exclusion and inclusion criteria in order to better determine the proper treatment of disorders including dorsonasal targeted therapies.

### RELATED APPLICATIONS

This application is a divisional of U.S. Ser. No. 16/551,657 filed on Aug. 26, 2019, which claims priority to U.S. Provisional Patent Application Ser. No. 62/722,778, filed Aug. 24, 2018; Ser. No. 16/551,657 is a continuation-in-part of U.S. Ser. No. 15/998,669 filed on Aug. 15, 2018 all of which are incorporated herein in their entirety as if set out in full.

### BACKGROUND OF THE INVENTION

Headache, facial, or head pain can be treated with medications delivered orally, via injection, patch, spray, or nasal modalities. Nasal delivery may target the SPG or other dorsonasal structures. Similarly, topical patches may be placed at various loci. Stimulation may be applied simultaneously to a variety of loci.

There is current controversy as to efficacy of transnasal or topical application of agents, including local anesthetic agents, Botox, or other medications or compounds to treat headache, facial or head pain or other symptoms applied to modulate the SPG or other dorsonasal structures.

Said treatments are effective.

However, there are multiple reasons why such treatments may lack efficacy. These include suboptimal delivery to the targeted structure, the presence of overlying adhesions, scar, cysts or other structures, mucosal hypertrophy, tumor, mass, mucous, dried mucous, inflammation or debris. In the case of SPG block, inadequate local anesthetic concentration or volume. 1 ml or less, 2 ml or less, 3 ml or less, 4 ml or less 5 ml or less, inadequate concentration of substance, less than 0.5%, equal to or less than 0.75%, equal to or less than 1%, suboptimal placement i.e. not rotating head, or poor patient choice. Further inadequate surface area of delivery will decrease efficacy. In addition, pooling of agent in only one locus will not allow for optimal disbursing of agent.

The present invention classifies headache with an additional classification of patients to include not only “migraine” which may not be a migraine, or which may not delineate headaches as Local Anesthetic Responsive Headaches as a general class.

The present invention also uses exclusion and inclusion criteria in order to better determine the proper treatment of the headache.

### SUMMARY OF THE INVENTION

The present invention describes exclusion as well as inclusion criteria for selecting patient to be treated pharmacologically or with stimulation.

### DETAILED DESCRIPTION OF THE INVENTION

The exclusion criteria for selecting patients, such as no likelihood of Sinus related disorder; Initially, no Hx Sinus surgery, DNS surgery, no Positive Sinus “Tap Test” on exam; no occipital component, no triggering or out of range pain on exam of occipital nerves; no triggering or out of range pain on Cervical spine exam, no association with Cervical Spine or Brain Surgery; no TMD/TMJ component/Temporal major component; no triggering or out of range pain on TMJ or AT exam, or Temporalis muscle or Temporal artery exam; no Chronic Daily Headaches, migraines should be episodic; none of the Usual Psychologic contraindications present; would not include Fibromyalgia patients initially; unclear if current or prior opioid prescription use is a negative predictor; would consider substance abuse a negative predictor.

The present invention also describes inclusion criteria: presence of a major peri/retro orbital component indicates high likelihood of success; presence of visual changes ranging from loss of vision to just blurry vision, or the presence of scotomata are positive predictors of success; aura is a positive predictor of success; nausea/vomiting may be a positive predictor; the more severe the pain, the more dramatic the relief; failure of, or side effects from, Triptans and or antineuropathic agents is not a negative predictor and may even have positive predictive value in some patients.

Additionally, a notably strong response to prior SPG block with longer acting agent is an important screening predictor. Repeated strong efficacy can be an indication of a successful stimulation result. Additionally, many blocks may not be done optimally and is another factor to be considered.

Failure to block a neural structure does not mean that neural blockade is ineffective. Success needs to be defined within the context of typical symptoms associated with a given headache or migraine subtype. Subsequent tension headaches in a patient stimulated for migraines is in no way a failure of stimulation therapy.

Hence, patients who may have more than one type of headache-need to be able to separate symptomatologies of their experienced headache subtypes in order to disentangle non-migraine subtype symptoms from migraine symptomologies. This is not always easy, but can generally be done.

Multi locus stimulation or pharmacological therapy or block has significant benefits.

To optimize patient selection in treating headaches, there are currently in the neighborhood of 150 types of headaches recognized by the IHS in their International Classification of Headache Disorders, ICHD, essentially recognized as the official WHO sanctioned headache classification. The hierarchical classification divides HAs into major Categories: Primary Headaches; Secondary Headaches; and Cranial Neuralgias, Central and Primary facial pain, and Other headaches.

These classifications are fairly specific, and in the development of effective remedies it is extremely important to distinguish among headache type and subtype categories which are merely descriptive, symptomatic, or associative and those which more truly reflect actual pathophysiologic structural or functional pathways. Manipulations of the correct pathway or pathways involved in a disease process are often key to the effective treatment of problematic symptomatologies.

This point is quite apparent when addressing the lackluster presence of occipital stimulators in the treatment of Occipital Neuralgia. It became quite apparent to me that the actual presence of true ON amongst patients with complaints of occipital headache pain was much smaller than believed by the diagnosing Neurologists and other physicians. These headaches looked, and acted similar to ON, but had slightly different distributions, characteristics, pain qualities, responses to cervical motion, activity and exam findings. Predictably, the stimulation of the occipital nerves has not been very effective for the treatment. Occipitally located pain that was actually nonoccipital neuralgia. Further, there are really 3 branches of the ONs, and crossover is higher than C2, so some true ON patients did fail stimulation because of incomplete definition or logistic issues.

Many patients have failed stimulation had allegedly successful blocks of the occipital nerves. The reason for this can be understood by parallel models. For example, Topical Lidocaine is effective in decreasing knee, back or radicular pain not by penetrating and numbing the deep structures involved but by affecting central processing of pain by the brain. It is likely that by affecting rather superficial skin sensors in the general area of the pain, the brain effectively “pays less attention” to that small area of the humunculus involved and pain is lessened. It has been observed in patients who have L5-S1 radiculopathies, yet are misdiagnosed as having Sacroiliitis. Because a component of the pain of that Radiculopathy may manifest itself in a similar character and in the typical area of sacroiliac joint pain, injecting the joint often seems to decrease the pain of that Radiculopathy. However, the pain relief usually becomes shortly subpar in efficacy, and is less long lived, in general, than that obtained when one injects the SIJoint for true Sacroiliitis or the L5-S1 nerve root for L5-S1 Radiculopathy.

Hence, one good way to optimize stim efficacy for migraines is to select those patients with “home run” (i.e. decreased pain, photophobia, phonophobia, nausea and vomiting) type results of prior SPG block, not weakly or moderately positive results.

It is important to recognize that the mere diagnosis of migraine doesn't always mean it is a migraine. Hence sinusitis, which is commonly misdiagnosed as a neurologic headache, must be ruled out. This is difficult because oftentimes ENT specialists, armed with MRI or CT results to the contrary, are surprised by actual endoscopic significant findings for sinusitis or by positive responses to antibiotics. Clinically, a negative sinusitis history, or a history which is significant for the patient differentiating between a typical migraine and typical sinusitis headache would be helpful. MRI or CT showing thickened mucosa or evidence of sinusitis should be viewed as a potential negative predictor. A history of sinus or nasal surgery may be a negative predictor, unless nerve damage related to surgery is suspected. A positive sinus Tap Test is a negative predictor.

Further, the diagnosis of migraine itself may be problematic as it is often used as a wastebasket diagnosis by physicians and patients to describe a really severe headache. In fact, I personally like to think of a headache or migraine as a Local anesthetic responsive, or non-responsive headache. That is to say I define based upon whether the blockage of dorsonasal nerve structures stops an episode.

Also important is diagnosing other peripheral neuropathies which can look like migraines. Hence, a very positive Supratrochlear nerve, or other Tinel equivalent on exam, or long-lasting profound nerve block result probably should not have an SPG stim as monotherapy.

A history of occipital headache, crown headache, TMD, TMJ, Temporal arteritis tension type headache, and varying headache location are negative predictors. Chronic daily headaches generally don't respond to SPG stim. The presence of Nausea/vomiting, photophobia, phonophobia are not negative predictors, nor is high pain intensity.

These modalities may be used to treat pain, disease or suboptimal function involving any neurologic, intracranial, extracranial, nerve structure, spinal cord, cerebellar, pontine, Brainstem, basal ganglia, cranial nerve, or related structure, or peripheral nerve and particularly to treat of headache, cluster headache, migraine, facial pain, posttraumatic headache, Cervicogenic headache, occipital neuralgia, tinnitus, hearing or visual issues, postoperative or post-surgical head, neck or facial pain, Post herpetic neuralgia or to enhance recovery from reconstructive, or other surgeries from trauma, Oncologic, or deformative pathologies or states, to improve cognitive function, treat PTSD, treat psychiatric disorders, vascular dementia, Alzheimer's disease symptoms or cognitive decline, control dangerous individuals or to keep them within or away from certain locations, improve sleep hygiene, improve wakefulness, decrease unwanted sequelae of sleep deprivation and in the treatment of dysesthesias or dental or oral pain including but not limited to burning mouth syndrome.

One aspect of the invention relates to a method of inhibiting a disorder in a human patient, the disorder comprising method of inhibiting a disorder in a human patient, the disorder comprising pain, or loss of motor or sensory function, sympathetic tone or range or fluidity of motion that is not cerebral neurovascular disorder pain or not muscular headache pain (hereinafter jointly referred to as “non-CNvD or muscular headache pain”), the method comprising affecting a nerve pathway at one or more locus associated with the disorder in a manner to inhibit the disorder to thereby inhibit the disorder, wherein at least one locus is a peripheral nerve structure physiologically or anatomically related to the nerve pathway that directly targets a neural or other related structure associated with the disorder. This method is effective in treating various injuries, trauma, post-surgical conditions, traumatic neuropathies and related disorders, including inhibiting pain associated with them, or for improving functionality following loss of motor or sensory function, sympathetic tone or range or fluidity of motion or trigeminal neuralgia or other disorder resulting from trauma, neoplasm, cancer, surgery, small fiber peripheral neuropathy or nerve damage or compromise, or sympathetic dysfunction involving the face, head, neck, back, oropharynx, oral, dental, temporomandibular joint or musculature (TMJ), thorax, abdomen, pelvis, genitalia, shoulder, back, elbow, wrist, fingers, hip, knee, ankle, toes or other joints, limbs or musculature or connective tissue or any combination of two or more of these disorders. This method comprises anesthetizing, blocking or disrupting at least at one locus a nerve structure, a small fiber branch or branches of one or more nerve structures or fibers involving sympathetic or parasympathetic one or more nerves that directly targets a neural or other related structure associated with the disorder to thereby inhibit the disorder. Any one or any combination of the Interventions could be used to do such anesthetizing, blocking or disrupting.

These modalities may be used to treat pain, disease or suboptimal function involving any neurologic, intracranial, extracranial, nerve structure, spinal cord, cerebellar, pontine, Brainstem, basal ganglia, cranial nerve, or related structure, or peripheral nerve and particularly to treat of headache, cluster headache, migraine, facial pain, posttraumatic headache, Cervicogenic headache, occipital neuralgia, tinnitus, hearing or visual issues, postoperative or post-surgical head, neck or facial pain, Post herpetic neuralgia or to enhance recovery from reconstructive, or other surgeries from trauma, Oncologic, or deformative pathologies or states, to improve cognitive function, treat PTSD, treat psychiatric disorders, vascular dementia, Alzheimer's disease symptoms or cognitive decline, control dangerous individuals or to keep them within or away from certain locations, improve sleep hygiene, improve wakefulness, decrease unwanted sequelae of sleep deprivation and in the treatment of dysesthesias or dental or oral pain including but not limited to burning mouth syndrome.

Another aspect of the invention is the combination of stimulation of SPG with branches of Trigeminal nerves, or occipital nerves. There is known C-2 and 3 connection with Trigeminal nerve and SPG and likely with C1. With injection of bupivacaine 0.5% to the Supratrochlear nerve, obtained better result with SPG block subsequently. With injection of occipital nerves with bupivacaine 0.5%, headache was further decreased with SPG block.

Another aspect is the combined dorsonasal neural structure including one or more of the SPG, the cavernous sinus ganglion, the cerotic sinus ganglion, numerous branches of the maxillary nerve, the ethmoidal nerve, the ethmoidal ganglion, and the vidian nerve or other neural structure in close proximity of the SPG or sphenopalatine recess with stimulation of occipital nerve or sub occipital or high cervical segment including any of Cervical level (or proximal) 1-3 stimulation. Combined stimulation of a dorsonasal nerve with any one or more peripheral branches of the Trigeminal nerve or facial nerve including but not limited to one or more of the superficial branches of the trigeminal nerve located extracranially in the face, namely the supraorbital, supratrochlear, infraorbital, auriculotemporal, zygomaticotemporal, zygomaticoorbital, zygomaticofacial, nasal and mentalis nerve, anywhere in the course of the facial nerve from the intracranial or cisternal segment distally to the temporal, zygomatic, buccal, mandibular and cervical branches including efferent motor, parasympathetic or afferent sensory branches of the facial nerve. Also, combined occipital nerve, suboccipital, or high Cervical segment stimulation with any 1 or more branches of the Trigeminal nerve or facial nerve including but not limited to 1 or more of the superficial branches of the trigeminal nerve located extracranially in the face, namely the supraorbital, supratrochlear, infraorbital, auriculotemporal, zygomaticotemporal, zygomaticoorbital, zygomaticofacial, nasal and mentalis nerve, anywhere in the course of the facial nerve from the intracranial or cisternal segment distally to the temporal, zygomatic, buccal, mandibular and cervical branches including efferent motor, parasympathetic or afferent sensory branches of the facial nerve.

The invention also comprises stimulation of one or more loci of the facial nerve or components thereof, with one or more electrodes or stimulator device component. Stimulators may be implantable or non-implantable.

These modalities may utilize an electrode or other stimulator device, magnet, heating device or component may be integral or detachable to a cap, hat, band, glass frame, mask, eye mask, sleep mask pillow, cushion or custom fit device apparatus for comfort and proper position. Optimal position may also be maximized by inflatable, temperature malleable or moldable bolsters or cushioning.

Multiple parameters may be independently adjusted for each stimulator locus including but not limited to timing of stimulation, synchronization or varied synchronization or nonsynchronization, waveform, frequency, amplitude, intensity, mode, directed stimulation and or signal propagation in anterograde or retrograde fashion or utilizing both in a manner including varied timing and parameters of antero and retrograde stimulation. In certain cases sensing patterns of neuronal conduction and generating augmented or subtracted stimulator induced propagation to cancel or resonate or otherwise alter conduction wave phases can decrease pain or improve and strengthen neuronal transmission to improve cognition, memory, motor coordination, sensation, or other functionalities in the CNS, PNS, musculoskeletal, endocrine or other organ or system function.

3 D or other neural mapping by XRay, MRI, CT, PET scan, ultrasound, or MR neurography will better elucidate neural anatomy, as well as the anatomy and ‘geometry’ surrounding or associated tissue and Boney structures and recesses to allow for improved formation, construction, and configuration of the stimulator or 1 or more components and optimize electrode or component placement and will facilitate better placement of electrode or stimulator lead. Mapping coverage of stimulation area, by noting geographic anatomic localization of stimulation paresthesia in healthy person, or in afflicted patient, will allow the map of stimulated area to be matched more closely with the patient's pain map and will optimize placement results. This can be done during placement, or adjusted for patient subgroup populations. Having ultrasound, XRay, MRI, ultrasound, or other CT or other modality to identify the stim or component will allow optimal placement. Hence radio opacity, or electrode or other localization modalities is productive.

One size, shape, array or configuration of stimulator type cannot deliver optimum stimulation for a given patient. Thin wafer or micro thin conductive electrodes may optimize placement.

3D printing of electrode or stimulator utilizing patient imaging studies will allow optimal peri or para neural placement. The array can be in three planes rather than a single lead point which is 2 dimensional or even linear. Based on actual imaging, a form of peri neural partial, or incomplete, garment type array would be optimum. Alternatively, different size stimulator arrays can be used to allow optimal patient placement by subgroup. Similarly, different shaped array configurations may be beneficial for different subgroups, and these can be based on population anatomic subtypes.

One method comprises intranasally administering a local anesthetic or local anesthetic pharmaceutical composition to a patient having non-CNvD or muscular headache pain in an amount effective to inhibit the pain or other symptoms of any of the disorders. According to this method, the local anesthetic pharmaceutical composition comprises a pharmaceutically acceptable carrier, at least one local anesthetic ingredient selected from the group consisting of a local anesthetic, a sustained release formulation of a local anesthetic or any effective medicament or compound and/or a compound selected from the group consisting of an anti-epileptic, phenytoin sodium, a benzodiazepam, ion, membrane stabilizing agent, a serotonin receptor agonist, a serotonin subclass 5HT1F receptor agonist, LY334,370, a sesquiterpene lactone, parthenolide, Tanacetum parthenium, an extract of Tanacetum parthenium, antineuropathic medication, gabapentin, pregabalin, duloxetine HCl, tricyclic or other antidepressant, amphetamine, ADHD medication, eszopiclone, muscle relaxant, zolpidem, sleeping agent, or cannabinoid agent.

Another aspect of the invention comprises anesthetizing a nerve structure, associated with the non-CNvD or muscular headache pain in the patient for a period effective to inhibit the pain. The nerve structure can be anesthetized by any method known in the art or described herein.

The present invention can also be applied to blocking more than one peripheral nerve associated with the Disorders of Interest via drug infusion or delivery.

Preferred nerve pathways to be treated are at least one of a nerve structure involving a suprascapular nerve or small fibers therein; a sympathetic or parasympathetic neural structure that is not directly located in the central nervous system; or stellate, lumbar paravertebral or other ganglia; a paraspinal branch of a neural structure; paraspinal sympathetic or parasympathetic fibers not in ganglion structures; a radicular nerve; or a small fiber or sympathetic or parasympathetic neural structure related to a major peripheral nerve. Other preferred nerve pathways to be treated are at least one of a surprascapular, radial, ulnar, median, musculocutaneous, a nerve of the lower extremities, such as tibial, peroneal, sural, saphenous or a nerve of the head and neck, such as a peripheral facial nerve.

Any one or any combination of the Interventions may be used to treat the locus or loci according to this embodiment of the invention, including but not limited to a one or more of a direct, a fanlike or a regional distribution of application of a Stimulation Technique to or administration of a pharmaceutical agent to small or peripheral nerve fibers associated with the nerve pathway.

Another aspect of the present invention relates to a method for treating a neuropathy associated with a disorder in a human patient, the method comprising: applying pressure in an increasing manner by palpation to an area associated with the neuropathy to determine a Keystone nerve which is triggering and essential to the neuropathy; applying the pressure

to determine a point of at least one of maximum discomfort or trigger of increased trophic symptoms or findings to identify neural or other related structure of initial intervention; and intervening to treat the neuropathy at the location of the neural or other related structure by one or more of foregoing Interventions, preferably by administration of a pharmaceutically active agent, internal implanted neurostimulation or external neurostimulation affecting a nerve pathway associated with the neuropathy in a manner to inhibit the neuropathy to thereby inhibit the neuropathy.

In this method, the neuropathy preferably includes but is not limited to neuropathy associated with a disorder selected from the group consisting of posttraumatic or postsurgical pain; cancer-related pain, peripheral neuropathy, trigeminal neuralgia, or loss of motor or sensory function, sympathetic tone or range or fluidity of motion of any one or more of the patient's face, head, neck, oropharynx, oral cavity, dental structure, temporomandibular joint or musculature, thorax, abdomen, pelvis, genitalia, joint, limb, musculature or connective tissue, such as tendons or ligaments, or any combination of two or more of the disorders.

Non-limiting, preferred disorders to be treated are those mentioned above regarding the treatment involving at least one, and preferably, at least two loci, as are the nerve pathways and nerve structures, so they will not be repeated here. Likewise, any of the Interventions may be used to treat the neuropathies or disorders identified using the neural or other related structure.

Many different types of interventions may be used to treat the disorders, neuropathies, indications, conditions and symptoms according to the present invention. Any single one or any combination of the disorders, neuropathies, indications, conditions and symptoms are treated according to this invention by affecting, such as by anesthetizing, blocking or disrupting a nerve pathway associated with the neuropathy, pain, disorder or dysfunction, such as but not limited to a dorsonasal nerve structure, a nerve of the head and neck, a nerve of the lower extremities, any portion of the spinal cord, surprascapular nerve, radial nerve, median nerve, ulnar nerve, musculocutaneous nerve, or peripheral or sympathetic nerves, including branches and small fibers of such nerves, associated with the neuropathy, pain, disorder or dysfunction in any manner to inhibit the neuropathy, pain, disorder or dysfunction. The nerve pathway may be anesthetized, blocked or disrupted by any of the following interventions: (a) performing acupuncture upon the nerve structure; (b) surgically intervening to disrupt or sever nerve structures; (c) by applying a Stimulation Technique or using a Stimulation Device as defined above, or (d) administering by any suitable means, such as parenterally, topically, transcutaneously, intranasally or dorsonasally, a local anesthetic or other pharmaceutically active agent capable of anesthetizing, blocking or disrupting any of the foregoing disorders, alone or together, to the area or areas containing or affecting the nerve pathway.

In addition to the local anesthetic, such pharmaceutical compositions may contain pharmaceutically acceptable carriers and other ingredients known to enhance and facilitate drug administration with the additional pharmaceutical agents disclosed herein. Compounds, formulations, and dosages of the additional pharmaceutically active agents described in this method are known in the art. Owing, in part, to the vasodilatory activity of local anesthetics, these compounds may be used according to this method at doses of about half their art-recognized doses to their full art-recognized doses.

Such pharmaceutical compositions may also contain ingredients to enhance sensory acceptability of the composition to a human patient, such as aromatic, aromatherapeutic, or pleasant-tasting substances. The pharmaceutical compositions may also, for example, be made in the form of a flexible solid or semisolid carrier comprising the local anesthetic, such as one of the carriers described in U.S. Pat. No. 5,332,576 or in U.S. Pat. No. 5,234,957; or in the form of suspended micro spheres, such as those described in U.S. Pat. No. 5,227,165. Solid and semi-solid formulations of some local anesthetics are preferred in the compositions, methods, and kits of the inventions, because such preparations improve local anesthetic localization. In these forms, there is less dilution of the local anesthetic by body fluids and less transport of the local anesthetic to an unintended body location. Furthermore, it is believed that these formulations will reduce or minimize unintended side effects such as disagreeable taste, oropharyngeal numbness, dysphasia, and compromise of protective reflexes. In these formulations, a lower amount of local anesthetic may be used, relative to other formulations.

Numerous pharmaceutically acceptable carriers are known in the art, as are methods of combining such carriers with local anesthetics. Examples of such carriers and methods are described, for example, in Genaro, ed., 1985, Remington's Pharmaceutical Sciences, Mack Publishing Co., Easton, Pa., and later editions.

It is understood that the pharmaceutical composition of the invention may comprise a combination of any of the forms described herein. By way of example, microparticles, microsomes, or liposomes comprising a local anesthetic may be suspended in a solution or other formulation of the same or a different local anesthetic, whereby the solution or other formulation provides a rapid onset of anesthesia and the local anesthetic in the form of microparticles, microsomes, or liposomes provides a sustained duration of anesthesia. Sustained release preparations may comprise a slowly-released formulation of a local anesthetic. Inclusion of another local anesthetic in such formulations, in a free or salt (i.e., not slowly-released) form confers to the formulation the ability to act both with a rapid onset of anesthesia and a sustained duration of anesthesia. All such combinations of formulations described herein are included in the invention.

The local anesthetic pharmaceutical composition useful for practicing the invention must be administered in a dose sufficient to inhibit pain associated with any of the Disorders of Interest for at least about one hour, and preferably for at least about two hours. Doses of the local anesthetic pharmaceutical composition may be administered in a single dose, in multiple doses, in sustained release doses, or continuously.

The local anesthetic(s) or other pharmaceutically active agent(s) may be present in the pharmaceutical composition at any concentration from a very dilute concentration through the solubility limit of the local anesthetic or pharmaceutically active agent(s) in the medium in which it is delivered. The local anesthetic(s) or other pharmaceutically active agent(s) may also be present at a concentration greater than the solubility limit of the local anesthetic or pharmaceutically active agent(s) in the medium in which it is delivered by using a crystalline, microcrystalline, or amorphous solid form of the local anesthetic, preferably suspended in a gel, foam, mousse, cream, liquid, liposome, microsome, solid polymeric matrix, or the like. In various embodiments, the local anesthetic may be administered in the form of a eutectic mixture of local anesthetics, such as described in U.S. Pat. No. 4,562,060, in the form of encapsulated or embedded local anesthetic, such as described in U.S. Pat. No. 5,085,868, in the form of an oil-in-water emulsion, such as described in U.S. Pat. No. 5,660,837, or in the form of an emulsion, a cream, a eutectic mixture, or a microemulsion, such as described in International Patent Application Publication No. WO 97/38675, particularly one having thermoreversible gelling properties. Because the nasal cavity is normally cooler than gum pockets, the environment disclosed in International Patent Application Publication No. WO 97/38675, a composition having thermoreversible gelling properties, wherein the composition is a fluid at about 20° C. and a gel or semisolid at the temperature in the human nasal cavity (i.e., about 30-37° C.), is preferred. Any of these compositions may be conveniently delivered dorsonasally and, once so delivered, will be available where placed within the nasal cavity for a sustained period after administration and will spread or drip into other tissues to a lesser degree than would a liquid composition. By using one of these formulations, less of the active compound yields greater therapeutic results and has significantly decreased side effects, such as local and systemic toxicity, tongue and oropharyngeal numbness, discomfort, bad taste, dysphasia, and possible compromise of protective airway reflexes.

Other possible formulations may be made by of one of skill in the art of pharmacology in view of this disclosure without departing from the spirit of the invention. See, for example, (Genaro, ed., 1985, Remington's Pharmaceutical Sciences, Mack Publishing Co., Easton, Pa., and later editions) for a number of forms of typical pharmaceutical compositions that may be adapted readily to the present invention in view of this disclosure.

Numerous pharmaceutically active agents are thought to exhibit their limited therapeutic activity by virtue of the ability of the agent to interact with one or more receptors present on the surface of cerebral blood vessels or other structures. By way of example, migraine therapeutic agents known as serotonin receptor agonists include such agents as sumatriptan and zolmitriptan, and are believed to interact with serotonin receptors. In order to exhibit their pharmacological effects, such agents must gain access by systemic vascular delivery to cerebral blood vessels which have altered vascular flow during an acute migraine episode (Scott, 1994, Clin. Pharmacokinet. 27:33 7-344) and must achieve a critical concentration at the cerebrovascular location of the corresponding receptor(s) in the compromised area. Thus, these pharmaceutically active agents must be administered at the onset of an acute migraine episode in order to avoid the cascade of inflammation that follows initiation of the episode (Limmroth et al., 1996, Curr. Opin. Neural. 9:206-210). Following delivery of one of these agents to the compromised area of a cerebral blood vessel, the concentration of the drug gradually decreases at those sites, and rebound can occur.

Topical local anesthetics are vasodilators and therefore inhibit vasoconstriction, with the exceptions of cocaine, which is a vasoconstrictor. It is believed that the vasodilatory effects of topical local anesthetic administration results from both a direct effect of the anesthetic upon the affected blood vessel and from an indirect effect of the anesthetic upon nerve structures associated with the blood vessel.

1-17. (canceled)

18. A method of topically applying a device or therapeutic medical unit, comprising of placing one or more of a patch, protein, gelatin or polymer, semisolid containing a therapeutic agent, with an anatomical preformed shape, of one or more size or configuration, with adhesive base partially or completely covering said base, to assure good contact to, and prevention of movement from, an area overlying a head or facial locus overlying a peripheral nerve, a peripheral branch of a cranial nerve, neural foramen, or a head or facial muscle, or joint, injury site, site of fracture, burn site, site of infection, suture site or surgical area.

19. A method as in claim 18, where Topical application is overlying one or more loci chosen from the following nerves, or branches thereof, foramina, muscles or joints: a supratrochlear n., supraorbital n., infratrochlear n., infraorbital n., auriculotemporal n., an occipital n., a ganglion, an SPG, a trigeminal structure, one or more cervical facet joints, TMJ, temporalis muscle, forehead muscle or eyelid muscle.

20. A method as in claim 18, whereby the agent is comprised of or contains one or more of an ion, magnesium agent, silver agent, zinc agent, local anesthetic agent, botulinum toxin agent, calcium channel blocker, NSAID, acetaminophen, steroid, vasodilator, phosphodiesterase inhibitor or blocker, antibiotic, a tetracycline, clindamycin or like antibiotic, erythromycin or like antibiotic, cephalosporin or like antibiotic, Floxacin antibiotic, antifungal agent, Neosporin or triple antibiotic, bacitracin, antiviral agent, Acyclovir, gancyclovir, Anti HSV agent, Anti cold sore agent, vasoconstrictor, topical anti NGF agent, monoclonal antibody, toxin, amino acid, collagen, collagen precursor, neuron or myonutrient, hormone, estrogen, NO2, NO2 precursor or substrate, arnica agent, neutraceutical, CBD or other cannabinoid, ketamine, muscle relaxant, antineuropathic agent, gabapentenoid, cetyl myristoleate, cetylated fatty acid, fatty acid, vitamin, capsaicin agent, menthol, camphor, eucalyptus agent, herb, Vitamin, Vitamin C, Vitamin E, Vitamin D, anti-inflammatory agent, moisturizing agent, DMSO or related agent, transcutaneous transport agent, antidepressant agent, TCA, SSRI, Tramadol, heating agent, mechanically activated heating agent, cooling agent, mechanically activated cooling agent.

21. A method as in claim 18, whereby the therapeutic medical unit contains one or more of microchip, integrated circuit, internal or external battery or power source, intrinsic or extrinsic battery or power source, induceable power source, battery, activateable battery mechanism, magnet, electromagnet, induceable stimulator, induceable ultrasound component, electrostimulator, neurostimulator, heating unit, electrophoresis unit, liquid crystal, carbon nanotubule or structure.

22. A method as in claim 18, whereby one or more medical units may in the form of, or be physically associated with or attached to a Brace, cast, splint, dressing, bandage, a garment, face mask, hat, band, headband, wristband or brace, elbow garment or brace shoulder garment or brace, knee garment or brace, ankle band or brace, bandage, dressing, cast, or splint or any molded device.

23. A method as in claim 18, where the preformed unit is of a color or form to maximize invisibility or is colored and/or textured to blend in with a users surrounding skin.

24. A method as in claim 18, where the preformed unit is designed to deliver an agent and/or provide electric stimulation, magnetic energy or field, or heat, or cold to enhance beauty, or appearance or skin condition, or underlying muscle tone or to decrease swelling.

25. A method as in claim 18, where a treated disorder includes acne, rash, cold sore, genital herpes, shingles, postherpetic neuralgia, aphthous ulcer, gingivitis, toothache, burning mouth/tongue syndrome, post waxing pain, rash or inflammation, burn, abrasion, incisional or scar pain, infection, swelling or inflammation, insect, jelly fish, or other envenomation, poison ivy rash, rash, pruritis, arthritis, arthralgia, muscle spasm, tendinitis, ache, headache or contact dermatitis.

26. A device or therapeutic medical unit, comprising of one or more of a patch, protein, gelatin or polymer, semisolid containing a therapeutic agent, with an anatomical preformed shape, of one or more size or configuration, with adhesive base partially or completely covering said base, to assure good contact to, and prevention of movement from, an area overlying a head or facial locus overlying a peripheral nerve, a peripheral branch of a cranial nerve, neural foramen, or a head or facial muscle, or joint.

27. A device as in claim 26, where Topical application is overlying one or more loci chosen from the following nerves, or branches thereof, foramina, muscles or joints: a supratrochlear n., supraorbital n., infratrochlear n., infraorbital n., auriculotemporal n., an occipital n., a ganglion, an SPG, a trigeminal structure, one or more cervical facet joints, TMJ, temporalis muscle, forehead muscle or eyelid muscle.

28. A device as in claim 26, whereby the agent is comprised of or contains one or more of an ion, magnesium agent, silver agent, zinc agent, local anesthetic agent, botulinum toxin agent, calcium channel blocker, NSAID, acetaminophen, steroid, vasodilator, phosphodiesterase inhibitor or blocker, antibiotic, a tetracycline, clindamycin or like antibiotic, erythromycin or like antibiotic, cephalosporin or like antibiotic, Floxacin antibiotic, antifungal agent, Neosporin or triple antibiotic, bacitracin, antiviral agent, Acyclovir, gancyclovir, Anti HSV agent, Anti cold sore agent, vasoconstrictor, topical anti NGF agent, monoclonal antibody, toxin, amino acid, collagen, collagen precursor, neuron or myonutrient, hormone, estrogen, NO2, NO2 precursor or substrate, arnica agent, neutraceutical, CBD or other cannabinoid, ketamine, muscle relaxant, antineuropathic agent, gabapentenoid, cetyl myristoleate, cetylated fatty acid, fatty acid, vitamin, capsaicin agent, menthol, camphor, eucalyptus agent, herb, Vitamin, Vitamin C, Vitamin E, Vitamin D, anti-inflammatory agent, moisturizing agent, DMSO or related agent, transcutaneous transport agent, antidepressant agent, TCA, SSRI, Tramadol, heating agent, mechanically activated heating agent, cooling agent, mechanically activated cooling agent.

29. A device as in claim 26, whereby the therapeutic medical unit contains one or more of microchip, integrated circuit, internal or external battery or power source, intrinsic or extrinsic battery or power source, induceable power source, battery, activatable battery mechanism, magnet, electromagnet, induceable stimulator, induceable ultrasound component, electrostimulator, neurostimulator, heating unit, electrophoresis unit, liquid crystal, carbon nanotubule or structure.

30. A device as in claim 26, whereby one or more medical units may in the form of, or be physically associated with or attached to a Brace, cast, splint, a garment, face mask, hat, band, headband, wristband or brace, elbow garment or brace shoulder garment or brace, knee garment or brace, ankle band or brace or any molded device.

31. A device as in claim 26, where the preformed unit is of a color or form to maximize invisibility or is colored and/or textured to blend in with a users surrounding skin.

32. A device as in claim 26, where the preformed unit is designed to deliver an agent and/or provide electric stimulation, magnetic energy or field, or heat, or cold to enhance beauty, or appearance or skin condition, or underlying muscle tone or to decrease swelling.

33. A device as in claim 26, where a treated disorder includes acne, rash, cold sore, genital herpes, shingles, postherpetic neuralgia, aphthous ulcer, gingivitis, toothache, burning mouth/tongue syndrome, post waxing pain, rash or inflammation, burn, abrasion, incisional or scar pain, infection, swelling or inflammation, insect, jelly fish, or other envenomation, poison ivy rash, rash, pruritis, arthritis, arthralgia, muscle spasm, tendinitis, ache, headache or contact dermatitis.

34-37. (canceled)
